(19) United States
(12) Patent Application Publication
(10) Pub. No.: US 2009/0209486 A1
(43) Pub. Date: Aug. 20, 2009

(54) Compositions of carbohydrates as dietary supplements

(76) Inventor: Roger Wayne Brown, Phoenix, AZ (US)

Correspondence Address: Roger W. Brown, Ph.D., 810 W. Villa Maria Dr., Phoenix, AZ, 85023, US

(21) Appl. No.: 12/079,907

(22) Filed: Mar. 31, 2008

Related U.S. Application Data
(63) Continuation-in-part of application No. 12/070,313, filed on Feb. 19, 2008.

(51) Int. Cl. A61K 31/70; A61P 3/02; A61K 31/715; A61P 25/28; A61K 31/726; A61K 31/716

(52) U.S. Cl. 514/54; 514/23

(57) ABSTRACT

In accordance with the present invention, there is to provide a dietary supplement that provides humans and animals with the essential carbohydrates needed to maintain proper health and functionality, to fend off illness, act as viral inhibitors, lessen the aging process of cells and to provide pets with another level of medications equal to that for humans.

### RELATED APPLICATIONS

This application claims priority on patent application Ser. No. 12/070,313 filed Feb. 19, 2008, and is a continuation-in-part of patent application Ser. No. 12/070,313 filed Feb. 19, 2008, the entire disclosure of which is incorporated herein.

The present application is related to U.S. Pat. No. 7,202,220 B2, issued Apr. 10, 2007, included by reference herein.

The present application is related to U.S. Pat. No. 7,199,104 B2, issued Apr. 3, 2007, included by reference herein.

The present application is related to U.S. Pat. No. 7,196,064 B2, issued Mar. 27, 2007, included by reference herein.

The present application is related to U.S. Pat. No. 7,157,431 B2, issued Jan. 2, 2007, included by reference herein.

The present application is related to U.S. Pat. No. 6,929,807 B1, issued Aug. 16, 2005, included by reference herein.

The present application is related to U.S. Pat. No. 3,890,438 A, issued Jun. 1, 1975, included by reference herein.

The present application is related to U.S. Pat. No. 3,947,601 A, issued Mar. 1, 1976, included by reference herein.

The present application is related to U.S. Pat. No. 4,260,603 A, issued Apr. 1, 1981, included by reference herein.

The present application is related to U.S. Pat. No. 4,466,958 A, issued Aug. 1, 1984, included by reference herein.

The present application is related to U.S. Pat. No. 4,777,045 A, issued Oct. 1, 1988, included by reference herein.

The present application is related to U.S. Pat. No. 4,871,557 A, issued Oct. 1, 1989, included by reference herein.

The present application is related to U.S. Pat. No. 5,612,039 A, issued Mar. 1, 1997, included by reference herein.

The present application is related to U.S. Pat. No. 5,827,526, issued Oct. 1, 1998, included by reference herein.

The present application is related to U.S. Pat. No. 7,244,706, issued Jul. 17, 2007, included by reference herein.

The present application is related to U.S. Pat. No. 7,323,179, issued Jan. 29, 2008, included by reference herein.

The present application is related to U.S. patent application Ser. No. 12/070,313 filed Feb. 19, 2008, for Compositions of Carbohydrates as Dietary Supplements, by Roger W. Brown, Ph.D.

### US Class: 514/8; 514/23; 514/54; 514/62; 424/725

### FIELD OF THE INVENTION

The present invention relates to the field of dietary supplements for mammals promoting good nutritional health, fending off illness and, more particularly, to the compositions of carbohydrates as dietary supplements that are required by mammals for optimum health.

### BACKGROUND OF THE INVENTION

Mammal's bodies produce a large number of different types of chemicals that the body uses to ward off disease, retard cell degradation, maintain memory and maintain overall body health. These chemicals are produced as a byproduct of what the mammal has eaten. If all of the right foods are eaten in the proper amounts then the body will produce enough of all of the chemicals required to keep it functioning properly. Over the years people have sought after which chemicals are actually necessary for good health and which ones are just good. As this field is evolving more and more information is being discovered about what chemicals mammal's bodies require for proper functionality.

Over the last ten years a lot of research has been done concerning cell communications and its importance to a properly functioning mammal's body. This research indicates that there are eight essential sugars that all mammals need in order to stay healthy; http://www.glyconutrients-center.org/ and http://www.glyconutrientsreference.com/. Six of these carbohydrates (sugars) are generally missing in the diets of most humans and seven are missing from the diet of most animals. However, very small concentrations of these missing carbohydrates are contained in various plants and some sea products. A synopsis of these essential sugars/carbohydrates and what functions they have been found to influence follows:

D-Galactose is readily available in human diets but not in most animal diets. It is obtained from the conversion of lactose (milk sugar) and is also easily obtained from dairy products unless you suffer from lactose intolerance or are a vegetarian who does not eat dairy products.

D-Mannose is not readily available in our diets. The most popular source is Aloe Vera. It is also available in tiny quantities in the bran of whole wheat. However, it is very unstable and must be taken fresh from the plant and properly standardized to be of any benefit. It plays a profound role in cellular interactions and has even been known to lower blood sugar levels. It is absolutely vital to proper immune defenses against microbial invaders and has a natural and powerful anti-inflammatory effect. This sugar is readily available in supplemental form. Good for: Wound healing, Diabetics, Anti-viral, Anti-inflammatory and Arthritis.

N-Acetyl-Glucosamine is not readily available in our diets. It is particularly beneficial for cartilage regeneration and joint inflammation. Glucosamine derivatives are well-known natural medicine for arthritic conditions comes from this sugar compound. It has many more therapeutic effects and deficiencies or malfunctions of this sugar have been linked to diseases of the bowel. Derivatives of this sugar are readily available in supplemental form. Good for: Wound repair, Range of motion, Insulin production, Arthritic conditions, Learning, HIV and Vision.

L-Fucose is not readily available in our diets but is readily found in breast milk, astragalus herb, in several medicinal mushrooms, and in certain brown algae. It has numerous well-documented benefits for the immune system and has been shown to inhibit some cancer growth and metastasis. Good for: Long term memory, Anti-viral, Cancer and tumors and Skin allergies.

D-Xylose is not readily available in our diets. It is often seen in sugarless gums, candies, etc. in that it has a sweet taste but does not cause tooth decay. It has recently been added to nasal sprays and appears to discourage the binding of allergens and pathogens to mucous membranes. It also has anti-bacterial and fungal properties and may help prevent certain cancers. Good for: Anti-fungal and gram negative bacteria.

N-Acetyl-Neuraminic Acid is not readily available in our diets but is another sugar that abounds in breast milk and dramatically impacts brain function and growth. It, too, boosts immune function and has documented anti-viral actions. Interestingly, in certain disease states the ability to digest this sugar is impaired. Good for: Anti-viral, Kidney stones, Asthma, Learning and Arthritis.

N-Acetyl-Galactosamine is not readily available in our diets. It is the least known of the essential sugars although it appears to inhibit the growth of some tumors and, like the other sugars, plays an individual role in keeping cellular messages clear and promptly delivered. Most of these sugars do not involve or require insulin for their use and go directly to the cells where they are incorporated into the cell structure wherever they are needed. Good for: Heart disease, Aging (cell rejuvenation), Joint functioning and Vision.

The actual body requirements for these missing carbohydrates has been hard to estimate because of their rarity and because of this the FDA has not set a lower daily intake limit on any of them. However, research has indicated a level of dose for each of these carbohydrates required to produce noticeable effects. The base level for each of these carbohydrates is about 0.005 mg/kg of body weight or 0.4 mg/day for a 150 pound mammal. These levels correspond to base levels of other medications.

The base level is not the required level to start seeing results but the level below which nothing much has been seen. The general therapeutic levels are above 0.1 mg/kg of body weight or 8 mg for a 150 pound mammal. These findings indicate that the minimum required level for these essential carbohydrates is a hundred times larger than is available in natural foods including the specially prepared supplements designed to alleviate these missing carbohydrate deficiencies.

The seven essential sugars/carbohydrates are: D-Galactose, L-Fucose, D-Mannose, D-Xylose, N-Acetyl-Glucosamine, N-Acetyl-Neuraminic Acid, and N-Acetyl-Galactosamine. Of these seven carbohydrates D-Mannose and Glucosamine derivatives are available as full strength supplements from a large number of over the counter drug stores and D-Galactose is available from specialty suppliers. The remaining four carbohydrates are much too expensive for companies to currently package in full strength so all that is generally available to the public are extremely low concentrations in food substitutes.

While there are several different companies selling glyconutrients only one of them has filed for patent protection in the US. This company is Mannatech, Inc. of Coppell Tex. and they sell their glyconutrients through a chain of 500,000 independent dealers worldwide. They filed an initial US patent application in 1997 and was granted a US patent on Aug. 16, 2005; U.S. Pat. No. 6,929,807 B1. Since then they have filed four additional filings to this one patent. They were granted additional patent numbers which are: U.S. Pat. No. 7,157,431 B2, U.S. Pat. No. 7,196,064 B2, U.S. Pat. No. 7,199,104 B2, and U.S. Pat. No. 7,202,220 B2.

Their first patent sets out eight essential sugars and ties these sugars to food sources where they can be found. However, they don't disclose the actual amount of the various essential sugars/carbohydrates in these food sources. Their web site is located at: https://www.mannatech.com/Default.aspx

They produce specific supplements used to address several different conditions that arise in mammals: weight management, alcoholism, nutrition, wellness management, lifestyle management, growth essentials, performance management, skin care and performance nutrition. All of these products are different mixes of the same basic foods, as seen above, and as can be seen they contain very little of what is required by the body to function properly.

Another company also selling glyconutrients is shown here: http://www.naturalcureguide.com/glyconutrients.html Again, as Mannatech does, this company also provides the greatest amounts of the carbohydrates that are not in short supply in the body.

While all of the companies selling glyconutrients have products specifically orientated to correct certain illnesses none of them have anything that addresses the common cold, cold remedies, Group 1 or Group 5 viruses, shingles, influenza, cold sores, oral ulcers, memory or cell aging, animal supplements. Clearly these missing carbohydrates have capabilities in all these areas but these areas are not being addressed by any of the current glyconutrient companies.

One of the short comings with the current offerings to the public is the lack of these missing carbohydrates at therapeutic dose levels (levels at which changes are seen in hours instead of many months or years). For example, L-Fucose is available in Gum Tragacanth ($34/pound) and Brewer's Yeast (half a pound for $6), but the availability of L-Fucose in Gum Tragacanth is only 0.1% by weight and only 0.05% in Brewer's Yeast. Currently available glyconutrient supplements supply less than 0.1 mg of L-Fucose per daily dose.

N-Acetyl-Galactosamine is available in shark cartilage (3 oz. $16) but there is only 0.01% by weight of it there. By taking the specially made glyconutrient supplements available from the above companies the daily dose of N-Acetyl-Galactosamine is still generally under 0.1 mg. Likewise, N-Acetyl-Neuraminic Acid is available in Whey Protein (36 26 gram servings for $33) and Hen's eggs but there is only 0.02% of it there by weight. A daily dose from one of the special glyconutrient supplements generally has less than 0.2 mg of it available.

Mannatech even admits that their formulations are extremely weak and require many months to see any results at all “Be patient! Research shows that it may take up to 4 months (or more) to notice the effects of any changes you make to your diet.” https://www.mannatech.com/Shopping/Product.aspx and also see https://www.mannatech.com/Shopping/RDReports.aspx

There have been numerous complaints that Manntech products do not provide any benefit at all even after months of usage; http://www.reviewcentre.com/reviews274152.html “I saw Mannatech Ambrotose, Glyconutrients advertised on a Fibromyalgia site. I was contacted by a Mannatech Associate and was advised that Ambrotose could help with all my ailments—Fibromyalgia, Osteoarthritis, Asthma, allergies etc. I was also told that I wouldn't need to continue to take the vitamin, mineral and herbal supplements I had been using. Now, 4 months on, my asthma has worsened, my cholesterol levels have gone up and my fibromyalgia and arthritis are unchanged.”

From this it can be seen the currently available special glyconutrient supplements sold to replace these missing carbohydrates contain mainly filler material and other chemicals that are already available to the body through other sources or are not needed by the body at all. Additionally, many of these special supplements contain ingredients known to excite an allergic reaction in a large part of the population; such as Whey Powder and Aloe Vera ingredients. On the other hand there is no known allergic reaction to these essential sugars when taken in their pure form even at therapeutic dose levels.

One of the essential sugars (a carbohydrate), N-Acetyl-Neuraminic Acid (Sialic Acid), has been shown to be more than 1000 times more effective at killing viruses than any other known medication when used in therapeutic doses “Another study reported in Antimicrobial Agents & Chemotherapy, November 1995, p. 2583-2584, stated that a sialic acid mixture was up to 1000 times more effective in fighting influenza types A and B, a Group 5 virus, than potent antiviral drugs. http://www.glyconutrients-center.org/N-acetylneuraminic-acid.php

Viruses also cause cold sores (also called fever blisters), oral ulcers, herpes simplex, hepatitis, viral pneumonia, smallpox, Ebola, rabies, as well as the common cold, to mention a few. Other essential sugars have the ability to fight off Group 1 viruses and they are as good at this as Sialic Acid is with Group 5 viruses. However, no one has commercialized these benefits of the essential sugars.

Even though the effectiveness of Sialic Acid as an antiviral agent been known for over a decade, Antimicrobial Agents & Chemotherapy, July 1993, p. 1473-1479, it is still waiting to be offered to the public in therapeutic dose levels. Its been found that derivatives of Sialic Acid have been shown to be hundreds of times more effective than Sialic Acid alone and a few have been commercialized, Antimicrobial Agents & Chemotherapy, October 1994, p. 2270-2275, and are selling abroad. Silaic Acid when used alone is effective at fighting certain Group 5 viruses but it is a much better antiviral agent when employed as one of its derivatives or when used in conjunction with other compositions.

It is therefore an object of the invention to aid the body's ability to fight colds and treat viral infections by providing it with the correct mix and level of carbohydrates needed by the body to fend off viral infections and other disease.

It is another object of the invention to aid the body's ability to lessen the cells aging process by providing it with the correct mix and level of carbohydrates needed by the cells to keep them healthy and to keep the body's immune system healthy.

It is another object of the invention to give an animal the ability to fight viruses and infections much more effectively by providing it with the correct mix and level of carbohydrates needed by its body and afford the same level of health for them as afforded humans.

It is another object of the invention to promote good health and wellness to all types of mammals through the proper mix of carbohydrates targeted to specific aliments, like increasing mental ability and learning, wound repair and long term memory to mention a few of the benefits of these essential sugars.

### SUMMARY OF THE INVENTION

In accordance with the present invention, there is to provide a dietary supplement that provides a mammal with the essential carbohydrates needed to maintain proper health and functionality, to fend off illness, provide viral defenses, lessen the aging process of cells and to provide pets with an equal level of medications as that afforded to humans.

### BRIEF DESCRIPTION OF THE DRAWINGS

There are no drawings.

### DESCRIPTION OF THE PREFERRED EMBODIMENT

The carbohydrates included in the dietary supplement of the invention are available from a number of manufactures. Most are derived synthetically from other pure chemicals rather than being expensive plant or animal derivatives. A supplier for the two most expensive essential sugars, Sialic Acid (CAS#131-48-6) and N-Acetyl-Galactosamine (CAS#1811-31-0), is R&S PharmChem located in China http://www.rspharmchem.com. A supplier for L-Fucose (CAS#2438-804) is AppliChem located in Germany http://www.applichem.de/perl/catalog/catalog.pl. AppliChem can also supply two other more readily available essential sugars, D-Galactose (CAS#59-234) and D-Xylose (CAS#58-86-6). D-Mannose (CAS#3458-28-4) is available from a number of the larger supplement suppliers like NOW Foods http://www.nowfoods.com/. The remaining carbohydrate, N-Acetyl-Glucosamine (CAS#98632-70-3) is generally used in one of its many derivative forms. This invention uses the derivatives Glucosamine HCL (CAS#66573-21-5) and Glucosamine Sulfate (CAS#29031-194) instead of Glucosamine (CAS#3416-24-8). Both of these carbohydrates are readily available at drug stores. It should be recognized that the composition of the carbohydrate is not intended to be limited by the source from which it is obtained.

It should be stressed that this invention does not incorporate the use of Glucose or Acetylated Mannose. While Glucose is one of the eight essential sugars it is so prevalent in today's diets that adding additional amounts of Glucose in a supplement generally provides no useful benefit. Acetylated Mannose is a plant derivative from the Aloe Vera plant obtained during the purification process of Mannose from Aloe Vera. Acetylated Mannose has not been shown by independent research to be of any beneficial use as a dietary supplement.

Although the present invention includes the above cited seven essential sugars (carbohydrates), it should be noted that other carbohydrates, nutritional compounds or biologically active or inert compounds can be included in the dietary supplement of the invention. Such other ingredients may include spices, scents, thickening agents, emulsifiers, fragrances, flavorings, buffers, gels, binders, filler material, lubrication material, vitamins and or minerals and/or other such compounds that facilitate the formulation or administration of the inventive dietary supplement. These components can be provided separately to a mammal given said dietary supplement.

Many different types of vitamins and minerals can be included in the dietary supplement of the invention. While a few vitamins and minerals of synthetic origin do possess nutritional value, particular embodiments of the dietary supplement herein can contain nutritionally effective amounts of non-toxic vitamins and minerals obtained predominantly from natural sources.

Other compounds, agents and nutrients can also be included in the dietary supplement of the invention, for example: cellulose, calcium carbonate, stearic acid, amino acids, glycine, glycerin, glycerine, olive oil, essential fibers, essential oils, essential botanicals, essential enteric ecology, flora growth promoters, emulsifiers, essential fatty acids, fungi, fungal extracts, plant extracts, honey, scents, fragrances, and enzymes.

As all of seven of these essential sugars have a molecular mass well under 600 they can all be readily absorbed through the skin. This property allows these sugars to target areas that are very near the surface of the skin that would be difficult or impossible to reach by other means. To this end they could be made into pastes, saves and lotions and rubbed on the areas of the body where a particular surface or subsurface effect was desired.

Independent research indicates that these essential sugars are not stored in the body. After ingestion the sugars are assimilated into the blood stream within minutes (if taken orally on an empty stomach). Once in the blood stream they flow through the body and cells in need of these nutrients take what they need and the rest flows on. Most of these unused sugars are excreted via the urine within 12 hours after ingestion. The tests indicate that while excess of these sugars are excreted from the body within 12 hours the cells maintain an internal supply of these sugars for a period of up to a week. Studies have also shown that taking these seven essential sugars, in the levels covered by this invention, did not cause an abnormal rise in the blood sugar levels of diabetics.

The dietary supplement form of the invention has been prepared and human testing of it began in 2006. It can be administered to mammals in powdered form, reconstitutable powder, ingested powder, liquid-solid suspension, liquid, nasal spray, inhalation, hypodermic injection, skin patch, subcutaneous injection, paste forms, suave, capsule and tablet dosage forms. It should be readily obvious to one of ordinary skill in the science of formulations that the present dietary supplement can also be formulated appropriately for irrigation, ophthalmic, rectal, sublingual, transdermal buccal, vaginal, or dermal administration. Thus, other dosage forms such as chewable candy bar, concentrate, drops, elixir, emulsion, film, gel, granule, chewing gum, jelly, lotions, oil, paste, pastille, pellet, suave, shampoo, rinse, soap, sponge, suppository, swab, syrup, chewable gelatin form, or chewable tablet can be used.

Due to varying diets among people, the dietary supplement of the invention can be administered in a wide range of dosages and formulated in a wide range of dosage unit strengths. For example, for those people who are missing from their diet seven of these essential carbohydrates, a dietary supplement containing those carbohydrates in nutritionally effective amounts can be formulated. As well, for those people whose bioabsorption of essential carbohydrates is extremely efficient, a dietary supplement formulation containing reduced amounts of essential carbohydrates can be prepared.

It should be noted that the dosage of the dietary supplement can also vary according to a particular ailment or disorder that a mammal is suffering from when taking the supplement. For example, a person suffering from chronic colds will generally require a dose different than an animal would who is sick in order to obtain a benefit. An appropriate dose of the dietary supplement can be readily determined by monitoring patient response, i.e., general health, to particular doses of the supplement. As well, when another agent such as a vitamin and/or a herbal extract is being administered to a mammal along with the present carbohydrate dietary supplement, the appropriate doses of the supplement and each of the agents can be readily determined in a like fashion by monitoring patient response, i.e. general health, to particular doses of each.

It is contemplated by the invention that the dietary supplement can be administered simultaneously or sequentially in one or a combination of dosage forms. While it is possible and even likely that the present dietary supplement will provide an immediate overall health benefit, such benefit may take hours or days to materialize. Nonetheless, the present carbohydrate dietary supplement will provide a beneficial nutritional response in a mammal consuming it.

For the examples herein, the dietary supplement of the invention was administered as a powder-containing capsule. According to the capsule size and ingredients used in a given study exemplified herein, the dietary supplement was administered by oral ingestion. The indicated doses for humans in Example 1 are based upon #00 sized capsules.

### Example 1

A suitable composition for a product according to the present invention is shown in the following table.

| | | | Human |
|---|---|---|---|
| Carbohydrate | Weight % (range) | Weight % (tested) | (mg) |
| D-Galactose | 0.1 to 40 | 6.3 | 43 |
| L-Fucose | 0.1 to 90 | 3.1 | 21 |
| D-Mannose | 10 to 70 | 52.1 | 358 |
| D-Xylose | 0.1 to 70 | 6.3 | 43 |
| Glucosamine HCL | 10 to 60 | 25.1 | 173 |
| Sialic Acid | 0.1 to 50 | 6.5 | 45 |
| N-Acetyl-Galactosamine | 0.1 to 90 | 0.6 | 4 |

In this composition the ingredients Glucosamine HCL, D-Galactose and D-Mannose are optional and preferred. Instead of using Rice Flour as an inactive filler as is done in most products currently available to the public the three optional ingredients set out above were used as active fillers. A disadvantage of using Rice Flour as an inactive filler is its high glycemic index tends to drive a mammal's Triglyceride levels up very high, while using essential sugars as fillers doesn't.

The ingredients are typically in a powered form and are dry blended in a mixer. The mixture can then be packaged as a blended powder into capsules or caplets. In this example the mixture was packaged into size 00 capsules with an average weight of 687 mg for human doses and 25 mg for animal doses. The mg per ingredient for animals would be found by dividing the human ingredient dose in mg by 27.5, for example: for D-Mannose the animal ingredient dose would be 13.0 mg.

This composition is considered a health maintenance mix. It was designed to reduce the probability of infections, like colds, while taking the composition. There were three test trials run using this composition on both humans and animals. These three tests ran from late January 2007 to late December 2007. In the first test, that lasted for two weeks, a capsule/dose was administered twice a day for two weeks. The next test lasted for one month during which time one capsule/dose was administered per day at bedtime.

The third test lasted slightly over nine months and the dose was one capsule/dose a week administered at bedtime. During that time there were no deaths or adverse reactions to the composition by anyone in the test group either human or animal. Regular blood, lipid and electrolyte testing was done. A base line was run prior to the test and during the test blood testing was done regularly to determine if there were any adverse effects due to taking the composition.

The end result was that no one taking this composition contracted a cold or any other type of viral infection during the entire period of the test, even though one of the individuals in the test was prone to chronic colds and flu. This test extended through two different flu seasons and the test subjects continued to work everyday and come in contact with infected people on a daily basis yet they didn't catch anything while taking this composition. The blood tests didn't show any adverse effects from taking this composition.

In a follow up test these capsules were taken on a once a month basis by the same human test group from the first test, this time during the flu season. Within two weeks following a single monthly dose (this was during the four week pause after the end of the weekly tests) two of the subjects came down with a cold or flu. After this the test was stopped and no additional capsules were taken and within two weeks all the test subjects resumed their normal activity with no adverse or lasting effects.

During the animal tests old cats were exposed to new cats infected with FPV, an animal virus, on two occasions. Nothing special was done for the first exposure. The second time an additional dose was given them just after exposure. After these two exposures, which were several months apart, all of the old cats tested positive for FPV. However, none of the cats came down with any symptoms and are in excellent health at the time of this writing.

From this it should be obvious that this composition is also effective against animal viruses.

### Example 2

In a different embodiment of this invention another suitable composition for a product according to the present invention is shown in the following table:

| | | | Human |
|---|---|---|---|
| Carbohydrate | Weight % (range) | Weight % (tested) | (mg) |
| D-Galactose | 0.1 to 50 | 7.2 | 25 |
| L-Fucose | 0.1 to 90 | 14.4 | 50 |
| D-Mannose | 1.0 to 70 | 31.6 | 110 |
| D-Xylose | 0.1 to 70 | 10.8 | 38 |
| Glucosamine HCL | 1.0 to 50 | 10.8 | 38 |
| Sialic Acid | 0.1 to 50 | 10.8 | 38 |
| N-Acetyl-Galactosamine | 0.1 to 90 | 14.4 | 50 |

In this composition the ingredients Glucosamine HCL and D-Mannose are optional and preferred. In this composition D-Galactose is not considered to be optional as this composition is intended for animals as well as humans. This composition was packaged in a size #1 capsule and taken once a week.

This composition is considered a wellness mixture. It was designed to reduce the probability of infections, like colds, while taking the capsules. These ingredients provide Anti-Viral, Anti-Fungal and provide defense against Gram Negative Bacteria. The initial starting composition was derived from the findings of many independent researchers each working with just one of the essential sugars or their derivatives. The composition was then refined from the results of human and animal tests to the mixture shown.

In recent studies this composition was also found to be extremely effective at mitigating the effects of handover due to over consumption of alcohol by taking a single dose early in the morning.

### Example 3

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 90 | 22.4 | 75 |
| D-Mannose | 1.0 to 70 | 14.9 | 50 |
| D-Xylose | 0.1 to 70 | 14.9 | 50 |
| Glucosamine HCL | 1.0 to 50 | 3.0 | 10 |
| Sialic Acid | 0.1 to 90 | 22.4 | 75 |
| N-Acetyl-Galactosamine | 0.1 to 90 | 22.4 | 75 |

This composition was packaged in a size #1 capsule and taken twice at the onset of an infection. It is very effective for people who fly a lot, in this case they take a capsule a few hours before getting on the plane and it helps their body fight off the infections in the recycled cabin air especially during the flu season. This composition is good for both humans and animals.

This composition is considered a wellness plus mixture. It was designed to target Group 1 viruses. These ingredients also provide Anti-Viral, Anti-Fungal and provide defense against Gram Negative Bacteria. The initial starting composition was derived from the findings of many independent researchers each working with just one of the essential sugars or their derivatives. The composition was then refined from the results of human and animal tests to the mixture shown.

In another test a mixture of this composition was formed by combining two (2) parts of wellness plus powder with 0.5 parts of Olive Oil, then this was mixed into a paste. Then 8 mg of this paste was applied on an oral ulcer, also known as an aphthous ulcer, and within eight hours the ulcer had completely subsided.

Another paste effective against oral ulcers was made using 0.5 parts of Olive Oil, one (1) part honey and two (2) parts of the composition powder. These pastes were found to be extremely effective at treating oral ulcers in doses of 2 mg to 12 mg. All soreness and discomfort were generally gone within 20 minutes of application of the paste. Previously, there has been no treatment or remedy for oral ulcers and they had to run their course which generally lasts two weeks. These are thought to be caused by a virus related to the Herpes Simples virus.

These pastes are a very effective means of administering the invention in difficult places like the inside of the mouth where saliva will tend to wash the powdered compound away very quickly. The honey mix was shown to keep the compound in place more than five times longer than just the powder alone.

### Example 4

In a different embodiment of this invention another suitable composition for a product according to the present invention is shown in the following table:

| Carbohydrate | Weight % (range) | Weight % (tested) | Human (mg) |
|---|---|---|---|
| D-Mannose | 1.0 to 40 | 28.8 | 100 |
| Glucosamine Sulfate | 0.1 to 30 | 20.8 | 72 |
| D-Xylose | 0.1 to 70 | 28.8 | 100 |
| Sialic Acid | 0.1 to 50 | 21.6 | 75 |

In this composition the ingredient D-Mannose is optional and preferred. This composition was packaged in a size #1 capsule and taken twice a day when a viral infection is eminent. This composition is good for both humans and animals.

This composition is considered a cold pill mix. It was designed to reduce the effects of Group 5 viral infections, like colds, and speed recovery. These ingredients provide Anti-Viral and Anti-Fungal benefits except in this composition the amounts have been increased, over the wellness composition shown in Example 2, to achieve increased defense against Group 5 viruses.

### Example 5

In a different embodiment of this invention another suitable composition for a product according to the present invention is shown in the following table:

| Carbohydrate | Weight % (range) | Weight % (tested) | Human (mg) |
|---|---|---|---|
| D-Mannose | 1.0 to 70 | 42.5 | 145 |
| Glucosamine HCl | 0.1 to 30 | 6.2 | 21 |
| D-Xylose | 0.1 to 70 | 29.3 | 100 |
| Sialic Acid | 0.1 to 50 | 22.0 | 75 |

This composition was packaged in a size #1 capsule and taken twice a day when an infection is eminent.

This composition is considered a cold booster mix. It was designed to reduce the effects of viral infections and speed recovery. These ingredients provide Anti-Viral and Anti-Fungal benefits except in this composition the amounts have been changed, over the cold pill composition shown in Example 4, to achieve increased defense against a broader range of viruses.

Testing of this composition on mammals has shown it to be extremely effective at killing the common cold virus.

### Example 6

In a different embodiment of this invention another suitable composition for a product according to the present invention is shown in the following table:

| Carbohydrate | Weight % (range) | Weight % (tested) | Human (mg) |
|---|---|---|---|
| L-Fucose | 1.0 to 90 | 22.4 | 75 |
| D-Mannose | 1.0 to 90 | 25.4 | 85 |
| Glucosamine HCl | 0.1 to 30 | 9.0 | 30 |
| D-Xylose | 0.1 to 70 | 20.9 | 70 |
| Sialic Acid | 0.1 to 90 | 22.4 | 75 |

This composition is considered a cold booster plus mix and it was packaged in a size #1 capsule and two capsules were taken 12 hours apart when coming down with a viral infection. This composition was designed to target Group 1 viruses. It was tested and has been shown to be extremely effective at killing the Cold Sore and Shingles viruses, both are members of the Group 1 viruses. This composition also provides defense against Group 5 viruses and Gram Negative Bacteria. Examples of Group 1 viruses are: Smallpox, African Swine Fever, Herpes Simplex (Cold Sores, Fever Blisters and Genital Herpes), Chickenpox, Shingles and Mononucleosis. Gram Negative Bacteria are generally associated with intestinal disorders and infections such as Travelers Diarrhea.

While some of these diseases have vaccines for them the problem is that vaccines have to be developed for each specific mutation of the virus. Another concern specifically with the Smallpox virus is the fear that a designer variety will be produced as a weapon of mass destruction. If that were done the new strain would be impervious to all the current Smallpox medications known and a new treatment for it would have to be developed from scratch.

In this light one of the many novelties of this invention should become extremely evident, as these Sugars are coded for the specific nucleic acid of viral groups they could be used as bio-weapon defenses. This means that changing a virus's protein coating will have no effect on the ability of these Sugars to quickly kill the virus.

Tests of these compositions has shown that from ingestion to time to killing the invading virus is 30 minutes for a single area of the body, like a Cold Sore or Shingles, to under four hours for a whole body influenza type of infection.

When taken after the onset of a cold dramatic relief is felt within four hours of taking this composition. Two doses should be taken 12 hours apart and not within two hours of a meal. Generally 10 PM and 10 AM seemed to work best for the test subjects. In some cases just taking a single dose at bedtime was sufficient to completely end all of the symptoms by morning.

### Example 7

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 80 | 21.6 | 75 |
| Glucosamine HCL | 1.0 to 50 | 28.0 | 97 |
| Sialic Acid | 0.1 to 50 | 21.6 | 75 |
| N-Acetyl-Galactosamine | 0.1 to 90 | 28.8 | 100 |

In this composition the ingredient Glucosamine HCL is optional and preferred. This composition was packaged in a size #1 capsule and taken once a day. It is good for both humans and animals.

This composition is considered an anti-aging, learning and memory enhancer mixture. It is intended to reduce the effects of cell aging and enhance memory and learning ability. This composition will not reverse any current level aging effects already present but should help to reduce the rate at which the cells age progressively while taking this composition.

This composition has application as a treatment medication in learning disorders, memory disorders and age related disorders such as Alzheimer disease.

The initial starting composition was derived from the findings of many independent researchers each working with just one of the essential sugars or their derivatives. The composition was then refined from the results of human and animal tests to the mixture shown. Most of the research relating to aging using the essential sugars was done with animals.

### Example 8

In a different embodiment of this invention another suitable composition for a product according to the present invention is shown in the following table:

| | | | Human |
|---|---|---|---|
| Carbohydrate | Weight % (range) | Weight % (tested) | (mg) |
| D-Mannose | 1.0 to 80 | 9.7 | 35 |
| L-Fucose | 0.1 to 80 | 20.8 | 75 |
| Glucosamine HCL | 1.0 to 50 | 27.8 | 100 |
| Sialic Acid | 0.1 to 50 | 13.9 | 50 |
| N-Acetyl-Galactosamine | 0.1 to 90 | 27.8 | 100 |

This composition is considered an anti-aging plus mixture This composition was packaged in a size #1 capsule and taken once a week. It is intended to be used in conjunction with the wellness capsule where each are taken once a week three to four days apart.

This composition is designed to reduce the effects of cell aging and offer additional antiviral protection not available by taking the wellness capsule alone. This composition will not reverse any current level age effects already present but should help to reduce the rate at which the cells age progressively while taking the composition. This composition has ingredients that targets both Group 1 and Group 5 viruses.

This composition has been made into a lotion using Olive Oil and emulsifiers. This lotion is designed to be used on the face, neck and back of the hands, and other parts of the body to rejuvenate the cells close to the skin's surface. Year long tests have shown that the growth in the number, and size, of age spots on the back of the hands have not advanced since the start of using this compound. Tests are continuing to determine if use of this compound will reduce the number and or size of age spots already in place prior to the start of using this composition.

The initial starting composition was derived from the findings of many independent researchers each working with just one of the essential sugars or their derivatives. The composition was then refined from the results of human and animal tests to the mixture shown. Most of the research relating to aging using the essential sugars was done with animals.

### Example 9

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 90 | 28.8 | 100 |
| Glucosamine HCL | 1.0 to 50 | 42.4 | 147 |
| Sialic Acid | 0.1 to 60 | 28.8 | 100 |
| N-Acetyl-Galactosamine | 0.0 to 1 | 0.0 | 0 |

In this composition the ingredient Glucosamine HCL is optional and preferred. This composition was packaged in a size #1 capsule and taken once a day. It is good for both humans and animals. While this composition uses the same ingredients as Example 7 the amount of N-Acetyl-Galactosamine has been reduced to zero for this use.

This composition is considered a learning enhancer mixture. It is intended to increase the ability of one to learn new tasks and to improve memory. This composition has application in learning disorder diseases.

The initial starting composition was derived from the findings of many independent researchers each working with just one of the essential sugars or their derivatives. The composition was then refined from the results of human and animal tests to the mixture shown. Most of the research relating to learning ability using the essential sugars was done with animals.

It can be seen that this invention has a large number of possible beneficial compositions utilizing just one, or any combination, of the seven essential sugars specialized for a specific target. Just because a combination is not specifically set out herein should not limit the scope of this invention. It has been sufficiently shown that there are numerous compositions available with useful purposes by the detailed examples set out herein.

Additionally, the weighting of a composition ingredient will specify a new composition for a new target use even though the ingredients for two different target uses are the very same, refer to Examples 7 and 9. By changing the amounts of an ingredient its effects on cell absorption will change and by increasing or reducing an ingredient within a cell it will turn on or off different genes which will alter the body's response; The Geno Type Diet by Dr. Peter J. D'Adamo, Broadway Books, 2007, ISBN 978-0-7679-2524-2.

In summary, this invention pertains to the field of dietary supplements and nutritional support for promotion and maintenance of optimal good health. More specifically, the invention relates to compositions of seven essential sugars/carbohydrates as dietary supplements that are essential for a mammal's optimal health and functionality.

This invention will correct the problem caused by modern diets consisting of highly refined foods, from which many essential ingredients have been eliminated during processing, specifically the seven essential sugars needed for a properly functioning mammal. It will also cure the problem inherent in most of the glyconutrients available today that contain only trace amounts of these essential sugars while containing large amounts of inactive ingredients that can and do cause numerous allergic reactions with no benefit realized.

The above is a detailed description of particular embodiments of the invention. Those of skill in the art should, in light of the present disclosure, appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

Since other modifications and changes varied to fit particular operating requirements and environments will be apparent to those skilled in the art, the invention is not considered limited to the examples chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A composition of at least one, or more, carbohydrates selected from the following group: mannose, n-acetylneuraminic acid, fucose, n-acetylgalactosamine, xylose;
and
at least one, or more, selected from: glucosamine HCL, glucosamine sulfate, galactose, flavorings;
and
at least one, or more, selected from: honey, olive oil, glycerine, spices, a filler ingredient, a flowing agent, a lubricant, a binder, an emulsifier, fragrances.

2. The compositions of carbohydrates in accordance with claim 1, further comprising one, or more, non-toxic vitamins or minerals.

3. The compositions of carbohydrates in accordance with claim 1, further comprising one, or more, non-toxic herbal, fungal, plant or animal derived agents.

4. The compositions of carbohydrates in accordance with claim 1, used as an agent in combating viral organisms, to include: African Swine Fever, Herpes Simplex, Chickenpox, Ebola, Mumps, Measles, Rabies, Mononucleosis.

5. The compositions of carbohydrates in accordance with claim 1, used as a dietary supplement.

6. The compositions of carbohydrates in accordance with claim 1, used as an agent in treating Alzheimer disease, learning disorders or memory disorders.

7. The compositions of carbohydrates in accordance with claim 1, used as an agent in treating the common cold, the flu or influenza types A or B.

8. The compositions of carbohydrates in accordance with claim 1, used as an agent in treating fever blisters or cold sores.

9. The compositions of carbohydrates in accordance with claim 1, used as an agent in treating shingles or recurring shingles attacks.

10. The compositions of carbohydrates in accordance with claim 1, used as an agent in mitigating the effects of a hangover.

11. The compositions of carbohydrates in accordance with claim 1, used as a bio-weapon defensive treatment in combating designer variety viruses such as, but not limited to, Smallpox.

12. The compositions of carbohydrates in accordance with claim 1, used as an agent in treating oral ulcers.

13. The compositions of carbohydrates in accordance with claim 1, used as an agent in combating Group 1 or Group 5 viruses.

14. The compositions of carbohydrates in accordance with claim 1, used as an anti-aging lotion or supplement.

15. The compositions of carbohydrates in accordance with claim 1, that is an effective treatment against one, or more, of the following: gram negative bacteria, travelers diarrhea, intestinal disorders.

16. The compositions of carbohydrates in accordance with claim 1, administered to humans or animals using any of the following methods: ingested powder, capsule, caplet, tablet, liquid, lotion, paste, hypodermic injection, skin patch, subcutaneous injection, suppository, inhalation, nasal spray, suave, paste.

17. A composition of at least one or more carbohydrate(s) selected from the following group: galactose, mannose, n-acetylneuraminic acid, fucose, n-acetylgalactosamine, n-acetylglucosamine, glucosamine, xylose;
and
at least one, or more, selected from: glucosamine HCL, glucosamine sulfate, spices, scents, fragrances, emulsifiers;
and
is an effective treatment against one, or more, of the following: Group 1 viruses, Group 5 viruses, gram negative bacteria, flu, common cold, viruses, oral ulcers, Alzheimer disease, age related disorders, learning disorders, memory disorders.

18. The compositions of carbohydrates in accordance with claim 17, that is used as a treatment against one, or more, of the following: African Swine Fever, Herpes Simplex, Chickenpox, Shingles, Ebola, Mumps, Measles, Rabies, Mononucleosis, Smallpox, Influenza A and B.

19. The compositions of carbohydrates in accordance with claim 17, that is an agent used in treating animal viruses.

20. The compositions of carbohydrates in accordance with claim 17, that is an effective treatment against one, or more, of the following: gram negative bacteria, travelers diarrhea, intestinal disorders.
